(19) United States
(12) Patent Application Publication
(10) Pub. No.: US 2013/0144193 A1
(43) Pub. Date: Jun. 6, 2013

(54) HYDROGEL ULTRASOUND COUPLING DEVICE

(71) Applicants: George K. Lewis, JR., Ithaca, NY (US); JoAnne L. Guarino, Slaterville Springs, NY (US); Bryant Guffey, Ithaca, NY (US)

(72) Inventors: George K. Lewis, JR., Ithaca, NY (US); JoAnne L. Guarino, Slaterville Springs, NY (US); Bryant Guffey, Ithaca, NY (US)

(73) Assignee: ZETROZ LLC, Ithaca, NY (US)

(21) Appl. No.: 13/805,165

(22) PCT Filed: Jun. 24, 2011
(86) PCT No.: PCT/US11/41787

§ 371 Date: Feb. 20, 2013

Related U.S. Application Data
(60) Provisional application No. 61/358,336, filed on Jun. 24, 2010.

(51) Int. Cl. A61N 7/00

(52) U.S. Cl. CPC A61N 7/00
USPC 601/2

(57) ABSTRACT

The present invention relates to an ultrasound coupling device that includes a gel component and a coupling compartment. The present invention also relates to various kits and methods for using the ultrasound coupling device with low-intensity ultrasound transducers and therapy. The present invention also relates to methods of making the ultrasound coupling device of the present invention. The present invention further relates to an array that includes a plurality of ultrasound coupling devices of the present invention, and methods of using the array.

### CROSS-REFERENCE TO RELATED APPLICATIONS

This application claims priority benefit of U.S. Provisional Patent Application Ser. No. 61/358,336, filed Jun. 24, 2010, the disclosure of which is hereby incorporated by reference herein in its entirety.

### FIELD OF THE INVENTION

The present invention relates to an ultrasound coupling device, methods of using the device in various ultrasound applications, and methods of making the device.

### BACKGROUND OF THE INVENTION

Ultrasound technologies are used in a variety of imaging and therapeutic applications. For example, ultrasound is a widely recognized therapy used for the reduction of pain and inflammation and for acceleration of healing in patients with a wide range of injuries and other medical conditions. Until recently, the delivery of ultrasound therapy was limited to delivery by a medical professional in a professional healthcare setting. Smaller or more portable ultrasound devices (e.g., portable low intensity therapeutic ultrasound devices) can allow patients to self-administer ultrasound therapy outside the professional healthcare setting.

In ultrasound therapy applications, ultrasonic waves are produced by a transducer of a portable low intensity therapeutic ultrasonic device. The transducer is applied to the skin in the area of treatment. In order for the ultrasonic waves to leave the transducer and penetrate the skin, an acoustic gel has commonly been used as a coupling agent. The acoustic gel, which is applied between the target area, specifically the skin, and the transducer, tends to be applied in unmeasured amounts. Due to the unknown application amounts it is difficult to estimate the actual amount of acoustic energy that is delivered to the target area, and the efficiency of energy coupling from the transducer to the skin. Additionally, the current methods of applying the acoustic gel tend to be messy and inappropriate for patient self-administered low intensity therapeutic ultrasound treatment.

With the advent of patient self-administered low intensity therapeutic ultrasound, a method is required that assures the proper amount of an ultrasonic coupling agent is available between the transducer of the low intensity therapeutic ultrasound device and the target area and that such method of application of the coupling agent is sufficiently simple for a patient to use during the treatment period.

Further, therapeutic ultrasound devices are not able to be used for long periods, due to the non-portable size of the devices or the need for external power sources.

Previous attempts to provide bandages and other coupling devices for use with therapeutic ultrasound technologies have been reported. See, e.g., U.S. Pat. No. 4,787,888, U.S. Pat. No. 7,211,060, and U.S. Patent Application Publication No. US-2008/0200810. However, the ultrasound bandages or coupling devices provided in the art to date are insufficient for use with portable therapeutic ultrasound systems that are able to deliver ultrasound energy deep within tissue and that can be used for long periods of time.

There is also a need for ultrasound coupling devices that can be used with all types of ultrasound transducers, not just therapeutic ultrasound transducers, and that can enhance the efficiency of ultrasound transmission to a subject.

The present invention is directed to overcoming these and other deficiencies in the art.

### SUMMARY OF THE INVENTION

In one aspect, the present invention provides an ultrasound coupling device. In one embodiment, the ultrasound coupling device includes a gel component and a coupling compartment. The device is useful for all types of ultrasound applications.

In another aspect, the present invention provides a therapeutic ultrasound kit that includes an ultrasound transducer and an ultrasound coupling device of the present invention.

In another aspect, the present invention provides a method for performing physiotherapy on a subject. This method involves providing an ultrasound transducer contained within an ultrasound coupling device of the present invention, and applying therapeutic ultrasound energy to a subject, where the therapeutic ultrasound energy is generated by the transducer and emitted through the gel component of the coupling device.

In another aspect, the present invention provides a method for applying ultrasound energy to a subject. This method involves providing an ultrasound transducer contained within an ultrasound coupling device of the present invention, and applying ultrasound energy to a surface of a subject, where the ultrasound energy is generated by the transducer and emitted through the gel component of the coupling device. In one embodiment, applying the ultrasound energy to the surface of the subject is effective to alleviate pain in tissue of the subject in and around the surface.

In another aspect, the present invention provides an ultrasound coupling device as described herein.

In another aspect, the present invention provides methods of using the ultrasound coupling device of the present invention, with the methods being as described herein.

In another aspect, the present invention provides methods of making the ultrasound coupling device of the present invention, with the methods being as described herein.

In another aspect, the present invention provides an array that includes a plurality of ultrasound coupling devices of the present invention. The array can be configured so that more than one ultrasound coupling device of the present invention is included in a holder component, where the holder component is configured to hold the plurality of ultrasound coupling devices in place on a surface of a subject. In one embodiment, the holder component can be configured as a wrap.

In another aspect, the present invention provides a method for applying ultrasound energy to a subject. This method involves (i) providing a plurality of ultrasound transducers contained within an array according to the present invention and (ii) applying ultrasound energy to a surface of a subject, where the ultrasound energy is generated by the plurality of transducers and emitted through the gel components of the plurality of coupling devices of the array.

In various other aspects, the present invention provides, for example, a disposable, window-framed hydrogel-based coupling device for use in low intensity ultrasonic therapy, and methods for use of the same, and more specifically, pertains to the use of a disposable window-framed hydrogel that remains stable and acoustically viable when transmitting low intensity ultrasound from a patient self-administered portable low intensity therapeutic ultrasound device to a therapy site, and a method for using the same.

These and other objects, features, and advantages of this invention will become apparent from the following detailed description of the various aspects of the invention taken in conjunction with the accompanying drawings.

### BRIEF DESCRIPTION OF THE DRAWINGS

For the purpose of illustrating aspects of the present invention, there are depicted in the drawings certain embodiments of the invention. However, the invention is not limited to the precise arrangements and instrumentalities of the embodiments depicted in the drawings. Further, as provided, like reference numerals contained in the drawings are meant to identify similar or identical elements.

FIG. 1 is an illustration showing a portable therapeutic ultrasound system that can be contained within the ultrasound coupling device of the present invention. The portable therapeutic ultrasound system shown in FIG. 1 includes ultrasound transducer 30 that is effective for wide-beam ultrasound therapy for extended pain relief. Cable 51 is shown attached to ultrasound transducer 30.

FIG. 2 is an illustration showing a portable therapeutic ultrasound system that is of a low-profile configuration and suitable for being integrated with the ultrasound coupling device of the present invention. As shown, lens component 70 and piezoelectric component 60 are assembled to form ultrasound transducer 50.

FIG. 3 is an illustration of one embodiment of the ultrasound coupling device of the present invention.

FIG. 4 is an illustration of a cross-sectional view of one embodiment of the ultrasound coupling device of the present invention.

FIG. 5 is an illustration of one embodiment of the ultrasound coupling device of the present invention coupled to a low-profile portable ultrasound transducer.

FIGS. 6A-6B are illustrations showing one embodiment of the ultrasound coupling device of the present invention. FIG. 6A shows a low-profile ultrasound transducer configured to snap into an ultrasound coupling device of the present invention. FIG. 6B shows the ultrasound coupling device of the present invention coupled to the low-profile ultrasound transducer shown in FIG. 6A.

FIG. 7 is an illustration of one embodiment of the coupling compartment of the present invention.

FIGS. 8A and 8B are photographs of one embodiment of the coupling compartment of the present invention.

### DETAILED DESCRIPTION OF THE INVENTION

The present invention relates to an ultrasound coupling device, as further described herein. The present invention also relates to various ultrasound kits and ultrasound transducer systems configured to include the ultrasound coupling device of the present invention. Further, the present invention relates to various methods of using and making the ultrasound coupling device of the present invention.

The ultrasound coupling device of the present invention has various attributes, as described more fully herein. Without meaning to limit the present invention to a particular embodiment, provided below are various attributes of the present invention.

The present invention provides a simple and disposable means to connect an ultrasound transducer or low profile ultrasound transducer or ultrasound therapy device to a specific region of a patient without having the need to manually hold the ultrasound transducer in place on the body. The invention makes the application of ultrasound therapy or ultrasound in combination with a topical pharmaceutical to be a simple and self-delivered process.

In one aspect, the present invention provides an affordable, highly adaptable and ergonomic means to secure ultrasound coupling gel to the face of an ultrasound transducer, and couple it to a patient or other object.

The ultrasound coupling device of the present invention may be used for ultrasound therapy, imaging, monitoring, industrial measurements and testing, anywhere ultrasound would be applied and requires attachment to some type of object or subject.

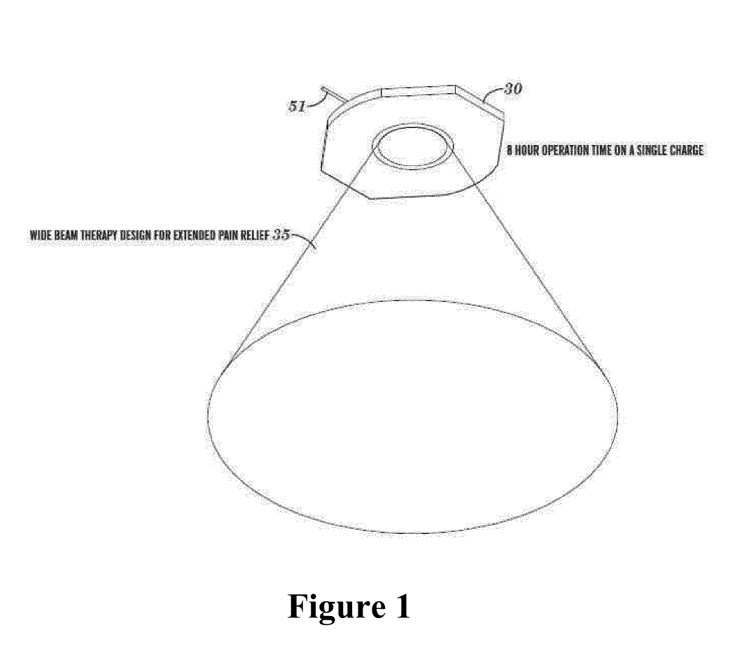

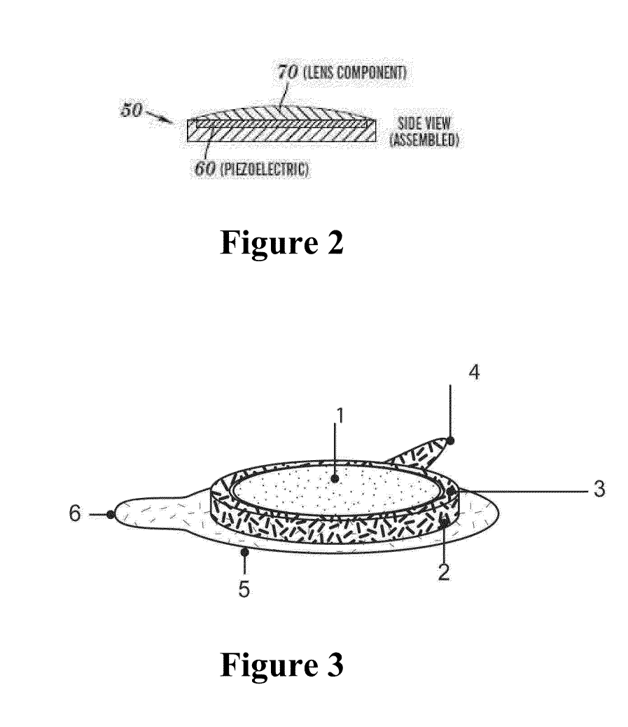

As referred to herein, the ultrasound coupling device may also be referred to as a specific embodiment for use as a hydrogel low-intensity ultrasound (LIUS) coupling patch device or variants thereof. However, the ultrasound coupling device of the present invention is useful for all types of ultrasound applications (e.g., imaging and therapeutic applications), and the gel is not limited to a hydrogel, but can include any type of gel or gel-like substance that can be used with ultrasound. Further, the ultrasound coupling device of the present invention can be used with various types of ultrasound transducers. In one embodiment, a suitable ultrasound transducer or ultrasound system for use with the ultrasound coupling device of the present invention can include, without limitation, a portable, low-profile type of ultrasound transducer (see, e.g., FIGS. 1 and 2).

Examples of portable ultrasound systems that can be used with the ultrasound coupling device of the present invention are provided in PCT/US2011/020061, the entire disclosure of which is incorporated by reference herein.

Examples of low-profile ultrasound transducers that can be used with the ultrasound coupling device of the present invention are provided in PCT/US2011/020062, the entire disclosure of which is incorporated by reference herein.

As provided herein, the gel component can be a hydrogel or any type of gel or gel-like substance that can be used with ultrasound. Therefore, in describing the various aspects and embodiments of the present invention, the term “hydrogel” can be used to refer to a hydrogel or any gel or gel-like substance that can be used with ultrasound.

In various embodiments, the gel component can be a hydrogel that is made of polymer materials that can absorb large amounts of water without dissolving due to physical or chemical cross-linkage of the hydrophilic polymer chains. Hydrogels which have low density cross-linking are more suitable conducting acoustic energy but low density cross-linking causes the hydrogel to be less ridged. The present invention is effective for using such hydrogels (as well as any other gel or gel-like material) for conducting acoustic energy from a low intensity ultrasound device to a subject.

In one aspect, the present invention provides a hydrogel LIUS coupling patch device that is designed to serve as an efficient acoustic conductive vehicle for the transmission of low intensity ultrasound between the portable low intensity therapeutic ultrasound device and the skin.

In one aspect, the present invention relates to the manufacture, composition, and use of biocompatible hydrogel acoustic coupling patches for transfer of low intensity therapeutic ultrasound to achieve pain relief, reduction of inflammation and healing.

In a particular embodiment, the hydrogel LIUS coupling patch device is constructed of a circular disk of a low density cross-linked hydrogel.

For example, the low density cross-linked hydrogel can be encircled on the outside by a window frame constructed of non-woven material. The non-woven frame surrounds the outer edges of the hydrogel in order to provide rigidity to the hydrogel allowing for a clear window area of the hydrogel for transmission of the ultrasonic waves from the transducer to the target area with minimal attenuation of the ultrasonic waves.

Although various embodiments describe a “non-woven” material or frame, the present invention is not meant to be limited to “non-woven” materials or frames, but also encompasses the use of any material that can function in place of the non-woven material or frame as described herein. Therefore, the descriptions below and elsewhere herein regarding a “non-woven” frame or material are not limited to non-woven materials, but are meant to include any such material that serves the same function as a non-woven frame or material.

The non-woven frame can have a low-tack adhesive on the outer side where the hydrogel LIUS coupling patch device affixes to the hydrogel LIUS coupling patch device.

The non-woven frame can have a low-tack adhesive on the outer side where the hydrogel LIUS coupling patch device affixes to the plastic coated applicator paper.

The hydrogel LIUS coupling patch device can be configured to provide acoustic impedance similar to that of tissue.

The hydrogel LIUS coupling patch device can be configured from biocompatible and latex free materials.

The hydrogel LIUS coupling patch device can be configured to have a low acoustic attenuation at low frequencies.

The hydrogel LIUS coupling patch device can be configured to be robust and durable during use.

The skin side of the framed hydrogel of the present invention can be seated on a protective plastic coated paper holder with a small flap for easy application and removal even for those individuals with reduced motor function of the digits.

The hydrogel LIUS coupling patch device can be configured to have a relatively low manufacturing cost.

The hydrogel LIUS coupling patch device can be disposable or non-disposable.

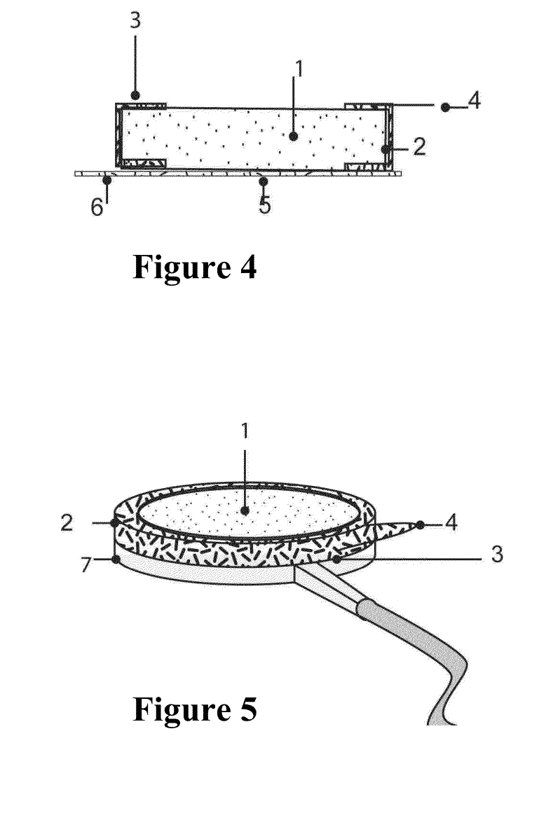

As shown in FIGS. 3 and 4, in one embodiment, the ultrasound coupling device of the present invention includes gel component 1 contained within coupling compartment 2. Coupling compartment 2 can optionally include tab 4 attached thereto for use in detaching the coupling device from attachment to an ultrasound transducer. A suitable gel component can be a hydrogel material that includes, without limitation, a low density cross-linked polymer hydrogel, which are well-known in the art. The present invention also includes the use of any type of medium that can transmit ultrasound and couple the transducer to the skin of a subject effectively. Therefore, the gel component need not be a hydrogel, and the hydrogel need not be a cross-linked hydrogel or a polymer. An adhesive material can be applied to surface 3 of coupling compartment 2. A suitable adhesive material can include, without limitation, a low-tack adhesive suitable to affix the coupling device to an ultrasound transducer and/or to the skin of a subject. Also shown is an optional applicator component 5 having an optional removal tab 6 for removing applicator component 5 from the ultrasound transducer, once the ultrasound coupling device is affixed to the ultrasound transducer.

As shown in FIG. 5, in one embodiment, the ultrasound coupling device of the present invention can be coupled with a low-profile ultrasound device 7.

The present invention can further be described as follows:

In one aspect, the present invention provides an ultrasound coupling device. In one embodiment, the ultrasound coupling device includes a gel component and a coupling compartment.

A suitable gel component can include, for example, a hydrogel material effective to conduct acoustic energy. In one embodiment, the hydrogel material, gel material, or gel-like material is effective to conduct acoustic energy across the entire therapy range, e.g., from about 10 to about 100,000,000 mW/cm². The acoustic energy can be in the form of low-intensity ultrasound waves. As stated above, the hydrogel material, gel material, or gel-like material is effective to conduct low-intensity ultrasound waves ranging from about 10 to about 100,000,000 mW/cm². The present invention also contemplates that suitable hydrogel materials, gel materials, or gel-like materials are effective to conduct low-intensity ultrasound waves at any value within the range of 10 to about 100,000,000 mW/cm². While not meaning to limit the present invention, examples of various suitable ranges of low-intensity ultrasound waves can include, without limitation, a range selected from the group consisting of between about 10 mW/cm² to about 50,000,000 mW/cm², between about 10 mW/cm² to about 1,000,000 mW/cm², between about 10 mW/cm² to about 500,000 mW/cm², between about 10 mW/cm² to about 250,000 mW/cm², between about 10 mW/cm² to about 100,000 mW/cm², between about 10 mW/cm² to about 50,000 mW/cm², between about 10 mW/cm² to about 40,000 mW/cm², between about 10 mW/cm² to about 30,000 mW/cm², between about 10 mW/cm² to about 20,000 mW/cm², between about 10 mW/cm² to about 10,000 mW/cm², between about 10 mW/cm² to about 6,000 mW/cm², between about 10 mW/cm² to about 5,750 mW/cm², between about 10 mW/cm² to about 5,500 mW/cm², between about 10 mW/cm² to about 5,250 mW/cm², between about 10 mW/cm² to about 5,000 mW/cm², between about 10 mW/cm² to about 4,750 mW/cm², between about 10 mW/cm² to about 4,500 mW/cm², between about 10 mW/cm² to about 4,250 mW/cm², between about 10 mW/cm² to about 4,000 mW/cm², between about 10 mW/cm² to about 3,750 mW/cm², between about 10 mW/cm² to about 3,500 mW/cm², between about 10 mW/cm² to about 3,250 mW/cm², between about 10 mW/cm² to about 3,000 mW/cm², between about 10 mW/cm² to about 2,750 mW/cm², between about 10 mW/cm² to about 2,500 mW/cm², between about 10 mW/cm² to about 2,250 mW/cm², between about 10 mW/cm² to about 2,000 mW/cm², between about 10 mW/cm² to about 1,750 mW/cm², between about 10 mW/cm² to about 1,500 mW/cm², between about 10 mW/cm² to about 1,250 mW/cm², between about 10 mW/cm² to about 1,000 mW/cm², between about 10 mW/cm² to about 750 mW/cm², between about 10 mW/cm² to about 500 mW/cm², between about 10 mW/cm² to about 250 mW/cm², between about 10 mW/cm² to about 200 mW/cm², between about 10 mW/cm² to about 150 mW/cm², and between about 10 mW/cm² to about 100 mW/cm².

A suitable coupling compartment can include, for example, a wall-like structure effective for holding the gel component in place. The wall-like structure can include a continuous or substantially continuous sidewall, a top surface for interfacing with a subject (e.g., a human's skin surface), and a bottom surface and/or side surface for interfacing with an ultrasound transducer. In a suitable configuration, the gel component is contained at least within a portion of the sidewall of the wall-like structure of the coupling compartment. However, the present invention also provides that the gel component or portion thereof can protrude from the coupling compartment before, during, or after coupling of a transducer to the ultrasound coupling device of the present invention.

The gel component can be a gel, a gel-like composition, a hydrogel, and the like. In one embodiment, the hydrogel material can be, without limitation, a low density cross-linked polymer hydrogel. Such suitable hydrogels are known in the art. In a particular embodiment, the gel component can be in the form of a wafer having any two-dimensional geometric shape. Suitable wafers for use in the present invention can have any size (e.g., thickness, surface area) suitable for transmitting ultrasound from the ultrasound transducer coupled to the wafer. In one embodiment, the wafer is configured so that it is effective to transmit ultrasound effectively from the entire face of the ultrasound transducer coupled thereto. In a particular embodiment, the wafer can have a thickness of between about 0.25 mm and about 5.0 mm, and a surface area to transmit ultrasound effectively from the entire face of the ultrasound transducer. Suitable examples of two-dimensional geometric shapes of the wafer can include, without limitation, a circle, oval, square, rectangle, triangle, pentagon, hexagon, heptagon, octagon, etc., and the like, including any arbitrary shape or moldable shape. The present invention further contemplates the suitable two-dimensional or three-dimensional geometric shapes of the wafer to further include any shape specifically molded to fit the face of any therapeutic or diagnostic transducer (e.g., for fetal monitoring, imaging, etc.).

In one embodiment, the coupling compartment is formed from a non-woven material. In a particular embodiment, the non-woven material is biocompatible. In another particular embodiment, the non-woven material latex-free. Various other suitable non-woven materials are known in the art and contemplated by the present invention.

In one embodiment, the coupling compartment can be configured to enable the operation of the ultrasound transducer when the ultrasound transducer is appropriately coupled to the coupling compartment. In such an embodiment, the coupling compartment can include an enabler mechanism or configuration so that the ultrasound transducer turns on or begins emitting ultrasonic energy only when it is correctly and securely fastened or coupled to the coupling compartment.

In one embodiment, the coupling device of the present invention can further include an adhesive material applied to the top surface of the sidewall for use in affixing the coupling device to the subject.

In one embodiment, the coupling device of the present invention can further include an adhesive material applied to the bottom surface of the sidewall for use in attaching a low intensity ultrasound transducer thereto.

In one embodiment, the coupling compartment can further include a tab attached thereto for use in detaching the coupling device from attachment to the ultrasound transducer.

In one embodiment, the coupling device of the present invention can further include an applicator component removably affixed to the bottom surface of the wall-like structure of the coupling compartment. The applicator component is useful for positioning the bottom surface of the coupling device to the ultrasound transducer. In a particular embodiment, the applicator component can be a plastic-coated or metal-coated paper applicator. The applicator component can be configured to include a removal tab for use in removing the applicator component after affixing of the ultrasound transducer to the bottom surface of the wall-like structure of the coupling compartment. In other embodiments, the applicator component can be affixed to the coupling compartment.

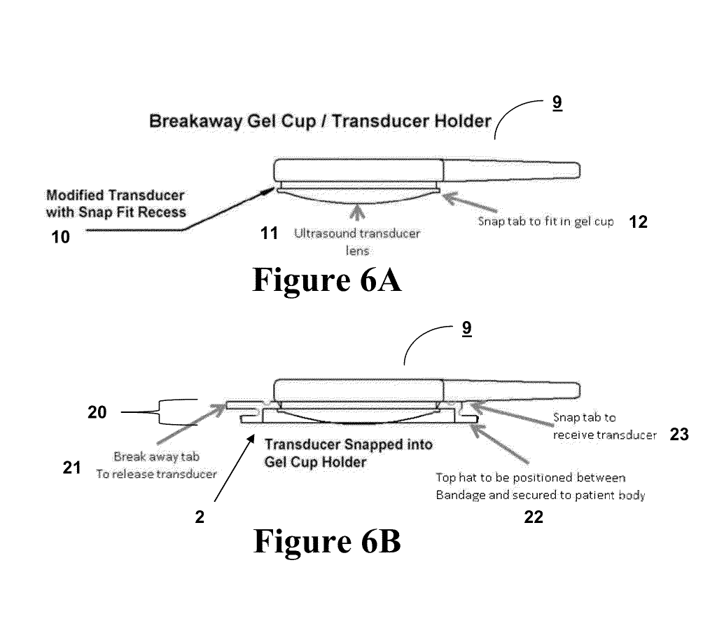

In one embodiment, the coupling device of the present invention is configured so that the coupling compartment can be removably coupled with an ultrasound transducer. Various such configurations are contemplated by the present invention, with certain of these configurations shown in FIGS. 6A, 6B, 7, 8A, and 8B and described below. In other embodiments, the coupling compartment can be affixed to the ultrasound transducer.

In a particular embodiment, the coupling device of the present invention can be configured to allow it to be used to hold a complete ultrasound system (i.e., if the system is coin sized or similarly small) by using the connection feature described herein below and shown in FIGS. 6A, 6B, 7, 8A, and 8B.

Transducer and/or ultrasound source/system modification: As shown in FIGS. 6A and 6B, in one embodiment, the ultrasound transducer or sound emitting portion of the LITUS system 9 has a modified snap fit recess 10 to receive the coupling device “referred to as the gel cup holder” (20). As shown in FIG. 6A, the transducer can be modified so that the ultrasound transducer lens 11 is configured with a snap tab 12 to fit into the gel cup holder (e.g., the gel component). The snap fit can be embodied in a number of ways with or without electrical connection or proprietary switching mechanism to allow activation of the LITUS system only when the device is positioned into the snap fit gel cup holder.

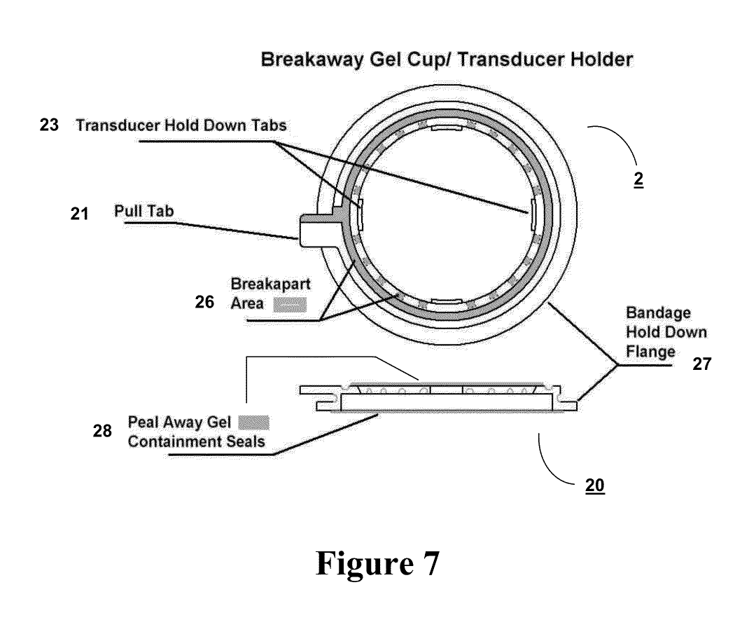

Transducer Gel Cup Holder: As shown in FIG. 6B, in one embodiment, the coupling device includes a reservoir for ultrasound coupling media, i.e., ultrasound gel, gel-like material, hydrogel, water, drug, or combinations thereof. As shown in FIGS. 6B, 7, 8A, and 8B, the gel cup holder can have a coupling compartment 2 that has tabs 23 that receive the snap recess fit tab 12 of the transducer 9 and may be allowed to activate the LITUS device. The tabs 23 of the gel cup holder's coupling compartment 2 are in one direction, so once the ultrasound transducer is “snapped in” it will not be easily removed from the gel cup holder. In one embodiment, as shown in FIG. 7, removal of the transducer from the holder can be accomplished by actually breaking the gel cup holder by pulling the pull tab 21 (e.g., a plastic tab) on the side (e.g., similar to a tab on a milk container before you open it), which will break the gel cup holder (see break apart area 26) and allow for easy removal of the ultrasound transducer from the coupling device. FIG. 7 also shows a side view of one embodiment of the ultrasound coupling device 20, which shows peal away gel containment seals 28. These containment seals are effective to keep the gel, gel-like material, or hydrogel contained within the coupling compartment (e.g., during storage prior to use). The containment seals can be removed to expose the gel and to allow for the coupling of the transducer to the ultrasound coupling device for use by a subject or patient, as described herein. The coupling compartment can include a bandage hold down flange 27, as shown in FIG. 7.

Although various embodiments of the ultrasound coupling device of the present invention can be configured to be disposable, the present invention also includes ultrasound coupling devices that are not disposable (also referred to herein as re-useable), in that they can be re-used and need not be destroyed after the ultrasound transducer is removed or de-coupled from the ultrasound coupling device. In a particular embodiment, the coupling compartment can be re-useable, while the gel component can be disposable. In another particular embodiment, the coupling compartment can be disposable, while the gel component can be re-useable. In another particular embodiment, the coupling compartment and the gel component are both re-useable. In another particular embodiment, the coupling compartment and the gel component are both disposable.

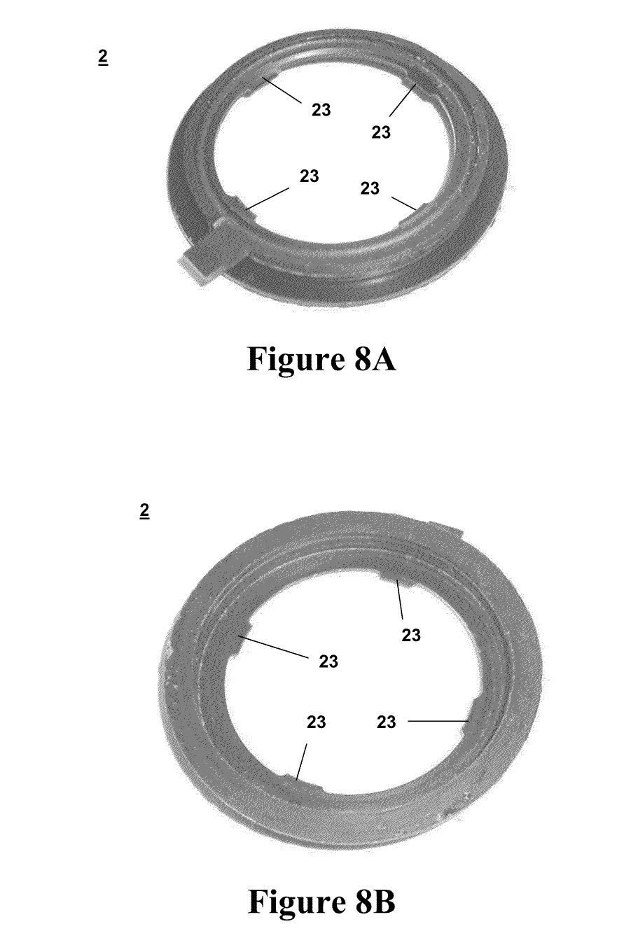

As shown in FIGS. 7, 8A, and 8B, in one embodiment, the coupling compartment includes hold down tabs or snap tabs. The transducer can be snapped into the coupling compartment and held at a certain distance from the body. The configuration shown in FIGS. 7, 8A, and 8B allows the transducer to “spin” while in the gel cup holder since it is only held in place by the tabs for forward and back motion, but not held in place for rotational motion. This will allow the user to position the transducer (and wire in some cases) in any rotational conformation on their body. The gel cup may be filled with any type of ultrasound coupling means and or drug. The gel cup may be made of any type of material, most likely the device will be made from soft injection moldable plastic to make the parts very low cost.

Important aspects of this configuration include the one-way securing of the transducer, and the removal of the transducer by using a pull tab in order to break away the transducer snap tabs or hold down taps away from the transducer itself. This break away feature allows for the easy removal of the ultrasound device from the bandage/gel cup holder, while also destroying the bandage/gel cup holder to make it only one time use.

The gel cup holder can have little wings on it at the bottom referred to as the “top hat” 22 in FIG. 6B. This “top hat” is to be secure between two bandage layers so the device may be secured to the skin. Thus, the gel cup holder is surrounded by a bandage to secure it in place (e.g., non-woven bandage, BAND AID® type, tegaderm, etc.). Another nice feature of the gel cup is that it has containment seals that may be removed before use by the subject. These seals hold the ultrasound coupling gel in place during storage of the device, thereby maintaining a long shelf life.

FIG. 8A is a photograph of one embodiment of the coupling compartment (i.e., a gel cup holder). FIG. 8A shows the top side of the gel cup holder that the transducer is inserted into. FIG. 8B is a photograph of the same embodiment of the coupling compartment, except it shows the bottom side, which is the side that faces the patient. These gel cup holders receive the transducer snap lip to secure it in place. These gel cup holders can be made of various types of materials, including, without limitation, such materials as ABS plastic, PVC, delyrine, and the like. As provided herein, these embodiments can be configured so that the gel cup holders are one time use, although they can also be configured so as to be re-used multiple times.

In another aspect, the present invention provides an array that includes a plurality of ultrasound coupling devices of the present invention. The array can be configured so that more than one ultrasound coupling device of the present invention is included in a holder component, where the holder component is configured to hold the plurality of ultrasound coupling devices in place on a surface of a subject. In one embodiment, the holder component can be configured as a wrap. Exemplary and suitable holder components and array configurations that can be used for the array of the present invention are provided in PCT/US2011/020052 (see, e.g., FIGS. 10, 11, 12, 13A, and 13B), PCT/US2011/020061 (see, e.g., FIG. 28C), and PCT/US2011/020062 (see, e.g., FIG. 28C), the entire disclosures of which are incorporated by reference herein. In another embodiment, the plurality of ultrasound coupling devices can be either of the same or different size, and can be of the same or different shape. Further, the ultrasound transducers used with the plurality of ultrasound coupling devices can be of the same or different size and shape, and also of the same or different ultrasound wave generation intensity. The ultrasound coupling devices can be configured to fit their particular corresponding ultrasound transducer.

In another aspect, the present invention provides a method for applying ultrasound energy to a subject. This method involves (i) providing a plurality of ultrasound transducers contained within an array according to the present invention and (ii) applying ultrasound energy to a surface of a subject, where the ultrasound energy is generated by the plurality of transducers and emitted through the gel components of the plurality of coupling devices of the array.

In one aspect, the present invention provides a therapeutic ultrasound kit that includes an ultrasound transducer and an ultrasound coupling device of the present invention. The ultrasound transducer can be coupled (e.g., affixed to) the ultrasound coupling device. In one embodiment, the ultrasound transducer is a low intensity ultrasound transducer. In another embodiment, the ultrasound transducer is a low-profile ultrasound transducer.

In one aspect, the present invention provides a method for performing physiotherapy on a subject. This method involves providing an ultrasound transducer contained within an ultrasound coupling device of the present invention, and applying therapeutic ultrasound energy to a subject, where the therapeutic ultrasound energy is generated by the transducer and emitted through the gel component of the coupling device.

In one aspect, the present invention provides a method for applying ultrasound energy to a subject. This method involves providing an ultrasound transducer contained within an ultrasound coupling device of the present invention, and applying ultrasound energy to a surface of a subject, where the ultrasound energy is generated by the transducer and emitted through the gel component of the coupling device. In one embodiment, applying the ultrasound energy to the surface of the subject is effective to alleviate pain in tissue of the subject in and around the surface.

While several aspects of the present invention have been described and depicted herein, alternative aspects may be effected by those skilled in the art to accomplish the same objectives. Accordingly, it is intended by the appended claims to cover all such alternative aspects as fall within the true spirit and scope of the invention.

What is claimed is:

1. An ultrasound coupling device comprising:
a gel component comprising a gel material effective to conduct acoustic energy; and
a coupling compartment comprising a wall-like structure effective for holding the gel component in place,
wherein said wall-like structure comprises a continuous or substantially continuous sidewall, a top surface for interfacing with a subject, and a bottom surface and/or side surface for interfacing with an ultrasound transducer, and
wherein said gel component is contained at least within a portion of the sidewall of the wall-like structure of the coupling compartment.

2. The coupling device according to claim 1, wherein said gel material is selected from the group consisting of a gel, a gel-like composition, a hydrogel, a low density cross-linked polymer hydrogel, and the like.

3. The coupling device according to claim 1, wherein said gel component is in the form of a wafer having any two-dimensional geometric shape.

4. The coupling device according to claim 3, wherein the wafer has a thickness of between about 0.25 mm and about 5.0 mm, and a shape to accommodate the entire face of the ultrasound transducer.

5. The coupling device according to claim 3, wherein said two-dimensional geometric shape can be of any shape, including an arbitrary or custom shape, or a shape selected from the group consisting of a circle, oval, square, rectangle, triangle, pentagon, hexagon, heptagon, octagon, and the like, or variations thereof.

6. The coupling device according to claim 1, wherein said coupling compartment is formed from a non-woven material or functionally similar material thereof.

7. The coupling device according to claim 6, wherein said non-woven material or said functionally similar material thereof is biocompatible.

8. The coupling device according to claim 6, wherein said non-woven material or said functionally similar material thereof is latex-free.

9. The coupling device according to claim 1 further comprising:
an adhesive material applied to the top surface of the sidewall for use in affixing the coupling device to the subject.

10. The coupling device according to claim 1 further comprising:
an adhesive material applied to the bottom surface of the sidewall for use in attaching an ultrasound transducer thereto.

11. The coupling device according to claim 1, wherein said coupling compartment further comprises a tab attached thereto for use in detaching the coupling device from attachment to the ultrasound transducer.

12. The coupling device according to claim 1 further comprising:
an applicator component removably affixed to the bottom surface of the wall-like structure of the coupling compartment, wherein said applicator component is useful for positioning the bottom surface of the coupling device to the ultrasound transducer.

13. The coupling device according to claim 12, wherein said applicator component comprises a plastic-coated paper applicator or a metal-coated paper applicator.

14. The coupling device according to claim 12, wherein said applicator component is configured to include a removal tab for use in removing the applicator component after affixing of the ultrasound transducer to the bottom surface of the wall-like structure of the coupling compartment.

15. The coupling device according to claim 1, wherein said coupling compartment is configured to removably couple with the ultrasound transducer.

16. The coupling device according to claim 15, wherein said coupling compartment comprises at least one tab component effective for removable coupling to the ultrasound transducer.

17. The coupling device according to claim 15, wherein said coupling compartment is configured for snap-down and twist-off coupling to the ultrasound transducer.

18. The coupling device according to claim 1, wherein said acoustic energy is in the form of low-intensity ultrasound waves.

19. The coupling device according to claim 18, wherein said low-intensity ultrasound waves are of a range of between about 10 mW/cm² and about 100,000,000 mW/cm².

20. The coupling device according to claim 1, wherein the coupling compartment is configured to enable the operation of the ultrasound transducer when the ultrasound transducer is coupled with the coupling compartment.

21. A therapeutic ultrasound kit comprising:
an ultrasound transducer; and
an ultrasound coupling device according to claim 1.

22. The kit according to claim 21, wherein said ultrasound transducer is a low intensity ultrasound transducer.

23. The kit according to claim 21, wherein said ultrasound transducer is a low-profile ultrasound transducer.

24. A method for performing physiotherapy on a subject, said method comprising:
providing an ultrasound transducer contained within an ultrasound coupling device according to claim 1; and
applying therapeutic ultrasound energy to a subject, wherein said therapeutic ultrasound energy is generated by the transducer and emitted through the gel component of the coupling device.

25. A method for applying ultrasound energy to a subject, said method comprising:
providing an ultrasound transducer contained within an ultrasound coupling device according to claim 1; and
applying ultrasound energy to a surface of a subject, wherein said ultrasound energy is generated by the transducer and emitted through the gel component of the coupling device.

26. The method according to claim 24, wherein applying the ultrasound energy to the surface of the subject is effective to alleviate pain in tissue of the subject in and around the surface.

27. An array comprising:
a plurality of ultrasound coupling devices according to claim 1; and
a holder component configured to hold the plurality of ultrasound coupling devices in place on a surface of a subject.

28. The array according to claim 27, wherein the plurality of ultrasound coupling devices are of either the same or different size and have either the same or different shape, and wherein the coupling compartment includes one tab to remove multiple transducers or a plurality of tabs to remove each transducer.

29. A method for applying ultrasound energy to a subject, said method comprising:
providing a plurality of ultrasound transducers contained within an array according to claim 27; and
applying ultrasound energy to a surface of a subject, wherein said ultrasound energy is generated by the plurality of transducers and emitted through the gel components of the plurality of coupling devices of the array.
